(19) United States
(12) Patent Application Publication
(10) Pub. No.: US 2020/0329842 A1
(43) Pub. Date: Oct. 22, 2020

(54) COSMETIC CASE UV STERILIZATION DEVICE

(71) Applicants: Su Chan LEE, Incheon (KR); Dong Gyu GONG, Incheon (KR); Jeong Mo KIM, Bucheon-Si Gyeonggi-do (KR); Yong Hwan KIM, Incheon (KR); GKIRI CO., LTD, Incheon (KR)

(72) Inventors: Su Chan LEE, Incheon (KR); Dong Gyu GONG, Incheon (KR); Jeong Mo KIM, Bucheon-Si Gyeonggi-do (KR); Yong Hwan KIM, Incheon (KR)

(21) Appl. No.: 16/960,887

(22) PCT Filed: Aug. 22, 2019
(86) PCT No.: PCT/KR2019/010671

§ 371 Date: Jul. 9, 2020

(30) Foreign Application Priority Data
Nov. 8, 2018 (KR) 10-2018-0136518

(51) Int. Cl. A45D 33/26; A45D 33/24; A61L 2/24; A61L 2/10; A61L 2/08

(52) U.S. Cl. CPC A45D 33/26; A45D 33/24; A61L 2/24; A61L 2/10; A61L 2202/14; A45D 2200/205; A61L 2202/11; A61L 2202/122; A61L 2202/121; A61L 2/088

(57) ABSTRACT

The present invention is directed to a cosmetic case, and relates to a UV sterilizing device of a cosmetic case that can sterilize the surfaces of a cosmetic material and a puff contaminated with sweat, dust, sebum, or bacteria, with a UV lamp. In the UV sterilization device, a lamp device portion (21) is provided on one side of the main body (10), the cosmetic material accommodation space (11) is disposed in a lower portion of a remaining side of the main body (10), the puff support plate (13) is disposed above the cosmetic material accommodation space (11), the puff support plate (13) is formed transparent or translucent, the cosmetic material accommodation space (11) is formed opaque, and UV lamps (20) are mounted to face from a side surface of the lamp device portion (21) to the bottom of the puff support plate (13).

### CROSS REFERENCE TO RELATED APPLICATIONS

This application is a national entry application of International Application No. PCT/KR2019/010671 tiled on Aug. 22, 2019, which claims priority to Korean Application No. 10-2018-0136518 filed on Nov. 8,2018. the entire contents of which are herein incorporated by reference in its entirety.

### TECHNICAL FIELD

The present invention is directed to a cosmetic case, and relates to a UV sterilizing device of a cosmetic case that can sterilize the surfaces of a cosmetic material and a puff, contaminated with sweat, dust, sebum, or bacteria during the use of the cosmetic case, with a UV lamp.

### TECHNICAL BACKGROUND

Cosmetic cases, such as compact foundations, generally include a body filled with a cosmetic material, a cover configured to be selectively opened and closed from the body, a mirror formed on the inner surface of the cover, a seating plate configured to allow a puffin the body to be seated thereon, and the puff configured to be seated on the seating plate.

Since such cosmetic materials contain moisture and puffs, which are cosmetic tools, are kept moist, various bacteria or bacteria are easily propagated in various sweats, dusts, sebum, etc. transferred from the users' skins, and the contaminated sides come back into contact with the users' skins, and thus a problem arises in that various skin diseases or troubles are caused.

Meanwhile, in order to solve the above-described problems, a sterilizing means for sterilizing a contaminated surface has been applied as in the following prior art document, and a UV lamp has been applied as a sterilizing means as in the present invention.

As an example of the prior art document to which the UV lamp is applied, in Korean Patent No. 10-0818024, a UV lamp is applied to the bottom of a cosmetic material inside a body. In this case, an actually contaminated surface is the surface of the cosmetic material. Accordingly, there is a problem in that it is difficult for the UV lamp disposed on the bottom of the cosmetic material in the body to reach the surface of the cosmetic material. Furthermore, there is also a problem in that the light of the UV lamp is radiated onto the bottom and side surfaces of the uncontaminated cosmetic material and the cosmetic material and the UV lamp are disposed close to each other, and thus the cosmetic material is dried and deteriorated by the heat emitted from the UV lamp.

As another example, in the case of Patent No. 10-1838571, UV lamps are configured to be directly disposed on the top of a cosmetic material and the bottom of a puff. As described above, a problem arises in that the cosmetic material is dried and deteriorated by the emitted heat of the UV lamps, and also problems arise in that it is difficult to install the UV lamps from the main body and it is difficult to apply power.

Furthermore, the above-described prior art documents have problems common that it is difficult to install the UV lamp, it is difficult to manufacture the cosmetic case, and it is inconvenient to use the cosmetic case.

For reference, the following prior art documents are the recent five prior art documents retrieved by using the keyword “cosmetic case*sterilization” through the patent information website “KIPRIS.” By reference to the following prior art documents, it will be possible to understand a technical field, a background art and development status related to the present invention.

### DETAILED DESCRIPTION

### Problems to be Solved

An object of the present invention is to basically sterilize a surface, contaminated during the use of a cosmetic case, with a UV lamp, as described in the background art section. More specifically, specific objects of the present invention are to sterilize a contaminated surface with a UV lamp as in the prior art so that only the contaminated surface can be intensively sterilized, to prevent the emitted heat of the UV lamp from being transferred to a cosmetic material, and to facilitate the mounting of the UV lamp and its related configurations, the manufacture of a cosmetic case, and the use of the cosmetic case.

### Solution to Solve the Problem

As a means for accomplishing the above object, there is proposed a UV sterilization device of a cosmetic case that is based on a cosmetic case including: a main body including a cosmetic material accommodation space filled with a cosmetic material, a puff support plate disposed above the cosmetic material accommodation space and mounted to be rotatable from one side of a top surface of the cosmetic material accommodation space, and a puff configured to be seated on the puff support plate; a cover mounted to be rotatable from one side of the main body and to cover the main body; and a mirror disposed on the inner surface of the cover, and

is characterized in that a lamp device portion capable of driving UV lamps is provided on one side of the main body, the cosmetic material accommodation space is disposed in a lower portion of the other side of the main body, the puff support plate is disposed above the cosmetic material accommodation space, the puff support plate is formed transparent or translucent, the cosmetic material accommodation space is formed opaque, and the UV lamps are mourned to face from a side surface of the lamp device portion to the bottom of the puff support plate.

### Advantages of Invention

According to a technical solution of the present invention, the UV lamp faces the bottom of the puff support plate and the puff support plate is formed transparent or translucent, so that the light of the UV lamp radiated from a side surface is guided through the puff support plate and is radiated onto the surface of the cosmetic material and the bottom of the puff. In this case, the cosmetic material accommodation space is formed opaque, and thus an effect is achieved in that only a contaminated surface is intensively sterilized compared to the prior art.

Furthermore, the UV lamp is disposed on a side surface, and thus the distance may be maintained between the UV lamp and the cosmetic material, thereby achieving the effect of preventing the drying and deterioration of the cosmetic material by preventing the emitted heat of the UV lamp front being transferred to the cosmetic material.

Moreover, the UV lamp is disposed on the side, and thus effects are achieved in that it is easy to mount the UV lamp and its related configurations, it is convenient to manufacture the cosmetic case, and it is easy to use the cosmetic case.

### BRIEF DESCRIPTION OF DRAWINGS

FIG. 1 is a perspective view of the present invention;

FIG. 2 is a perspective view of FIG. 1 with a puff support plate rotated upward;

FIG. 3 is a sectional view of FIG. 1;

FIG. 4 shows a perspective view (a), bottom perspective view (b) and plan view (c) of a state in which direct passage grooves and orthogonal grooves, are formed on the puff support plate of FIG. 1;

FIG. 5 is a partial sectional view to which FIG. 4 is applied;

FIG. 6 shows a perspective view (a), bottom perspective view (b) and plan view (c) of a state in which silver nano-pearls are formed on the puff support plate of FIG. 1;

FIG. 7 is a partial sectional view to which FIG. 6 is applied;

FIG. 8 shows a perspective view (a), bottom perspective view (b) and plan view (c) of a state in which direct passage grooves, orthogonal grooves, and silver nano-pearls are formed on the puff support plate of FIG. 1;

FIG. 10 shows a perspective view (a), bottom perspective view (b) and plan view (c) of a state in which light guide plates are formed on the puff support plate of FIG. 1;

FIG. 11 is a partial sectional view to which FIG. 10 is applied;

FIG. 12 shows a plan view (a) of a state in which direct passage grooves and orthogonal grooves are applied to FIG. 10 and a plan view (b) of a state in which diffusion patterns are applied;

FIG. 13 is a perspective view of a state in which a reflector is formed on the cover of FIG. 1;

FIG. 14 is a partial sectional view to which FIG. 13 is applied;

FIG. 15 is a perspective view of a state in which small holes are formed in the cover of FIG. 1;

FIG. 16 is a partial sectional view to which FIG. 15 is applied;

FIG. 17 is a perspective view of a state in which a detection sensor is applied to the lamp device portion of FIG. 1 and an overall perspective view of a state in which FIGS. 4 to 9 are applied to FIG. 1;

FIG. 18 is a sectional view of FIGS. 17; and

FIG. 19 is an operational block diagram of the lamp device portion of FIG. 17.

### EMBODIMENTS

The present invention will be described in detail below with reference to the accompanying drawings. However, the accompanying drawings may be exaggerated, omitted, or schematically illustrated for convenience of description regarding the main parts. The terms and names used in the description may be implicitly determined by the shapes, functions, or roles of configurations rather than the dictionary meanings. A description of a direction is determined based on the direction first presented in a first presented drawing. A description of an inner or outer location is determined based on the center of each configuration or the center of each configuration. Furthermore, detailed descriptions of the previously registered known technology and the conventional technology may obscure the subject matter, and may thus be omitted or replaced with simple symbols or names. Furthermore, the detailed structure, shape, pattern, arrangement, size, etc. of each configuration identifiable through a drawing, the operation of each configuration inferable through a drawing, and a resulting effect may obscure the subject matter, and thus detailed descriptions thereof may be omitted. Bolts, welds, holes, etc. applied for the coupling of configurations may obscure the subject matter, and may thus be omitted from the drawings. The shape of the plane of a configuration is circular or square unless the shape of the configuration is specifically specified, in which case the plan view may be omitted.

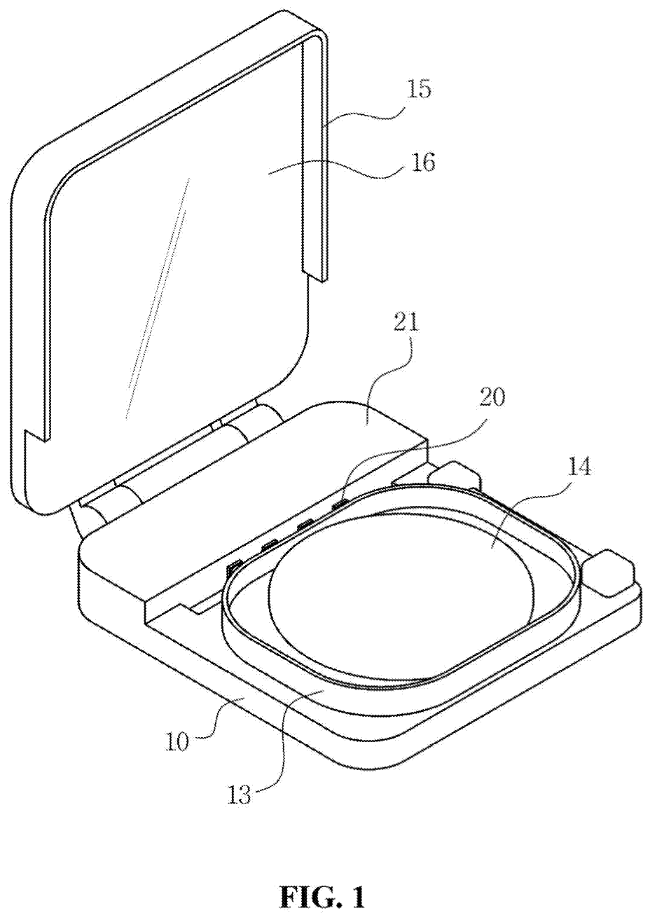

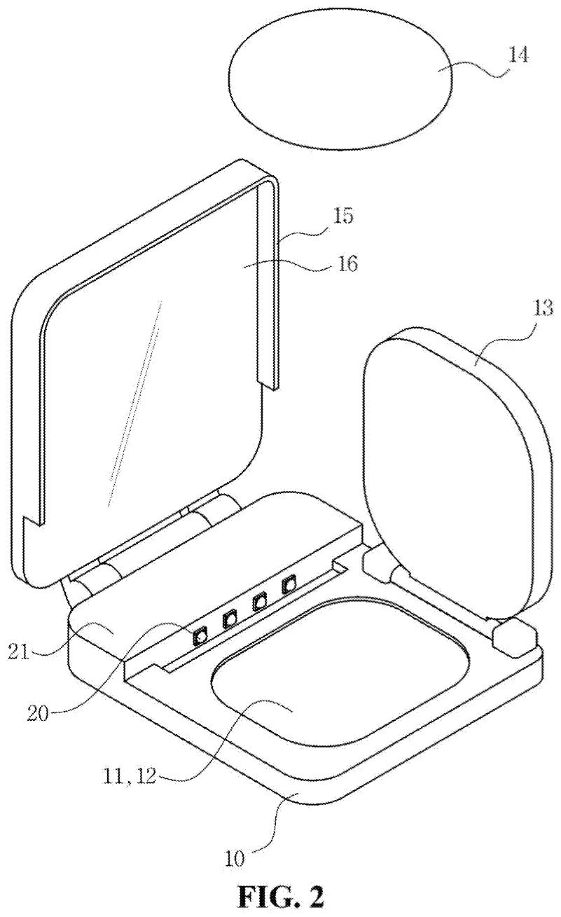

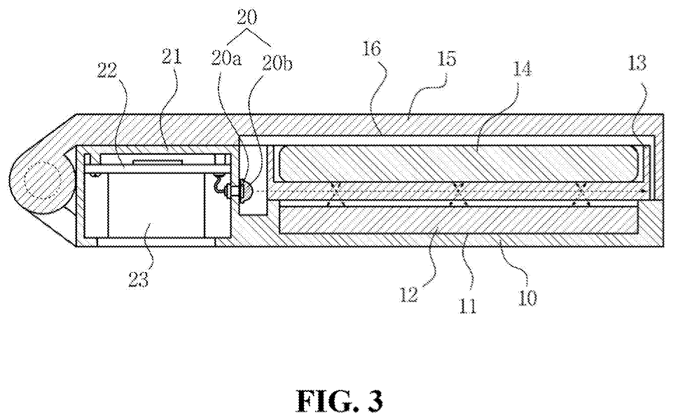

The present invention will be described with reference to FIGS. 1 to 3, as follows:

As shown in the drawings, the present invention is based on a cosmetic case including: a main body 10 including a cosmetic material accommodation space 11 filled with a cosmetic material 12, a puff support plate 13 disposed above the cosmetic material accommodation space 11 and mounted to be rotatable from one side of the top surface of the cosmetic material accommodation space 11, and a puff 14 configured to be seated on the puff support plate 13; a cover 15 mounted to be rotatable from one side of the main body 10 and to cover the main body 10; and a mirror 16 disposed on the inner surface of the cover 15, and

is characterized in that a lamp device portion 21 capable of driving UV lamps 20 is provided on one side of the main body 10, the cosmetic material accommodation space 11 is disposed in the lower portion of the other side of the main body 10, the puff support plate 13 is disposed above the cosmetic material accommodation space 11, the puff support plate 13 may be formed transparent or translucent, the cosmetic material accommodation space 11 is formed opaque, and the UV lamps 20 are mounted to face from a side surface of the lamp device portion 21 to the bottom of the puff support plate 13.

In other words, the lamp device portion 21 is added to one side of the main body 10, the cosmetic material accommodation space 11 and the puff support plate 13 over the cosmetic material accommodation space 11 are disposed on the other side, the UV lamps 20 are mounted to face the bottom of the puff support plate 13, the puff support plate 13 is formed transparent or translucent, and the cosmetic material accommodation space 11 is formed opaque.

In this manner, the light of the UV lamps 20 radiates the puff support plate 13, the puff support plate 13 is formed transparent or translucent, guides the light of the UV lamps 20 and radiates the light throughout the overall puff support plate 13 in a wide range, and the light is directly radiated onto the bottom surface of the puff 14 and the surface of the cosmetic material 12 corresponding to contaminated surfaces. In this case, the cosmetic material accommodation space 11 is formed opaque, and thus the light is radiated onto the surface of the cosmetic material 12, thereby achieving the effect of intensively sterilizing only the contaminated surfaces.

Furthermore, the UV lamps 20 are disposed beside the cosmetic material accommodation space 11 and the puff support plate 13, and thus a distance may be provided between the UV lamps 20 and the cosmetic material 12, so that the heat generated from the UV lamps 20 is prevented from being directly transferred to the cosmetic material 12, thereby achieving the effect of preventing the drying and deterioration of the cosmetic material 12.

Furthermore, the UV lamps 20 are disposed on the side, so that effects are achieved in that the UV lamps 20 and related configurations may be easily mounted, the cosmetic case may be easily manufactured, and convenient use may be achieved.

As shown in the drawings, the main body 10 may be formed in a rectangular shape, the lamp device portion 21 may be disposed on an inner surface where the cover 15 is rotated through a plane, the cosmetic accommodation space may be disposed on an outer surface, and the UV lamps 20 may be disposed along a side surface of the lamp device portion 21 at predetermined intervals.

Furthermore, the lamp device portion 21 may be provided with a substrate 22 therein, a battery accommodation portion 23 may be provided under the substrate 22, and the UV lamps 20 mounted on the side surface of the lamp device portion 21 may be driven by applying power from the substrate 22.

Meanwhile, the UV lamps 20 may include UV-LEDs 20a, as shown in the drawing. In addition, the UV-LEDs 20a may be covered with lenses 20b capable of narrowing the radiation angle of the UV-LEDs 20a.

Light is radiated from the UV-LEDs 20a at an angle of 120 degree. When the UV-LEDs 20a are covered with the lenses 20b, the angle at which light is radiated may be narrowed. This provides effects in that the light intensity may be made brighter in contrast to the same intensity, power consumption may be reduced by reducing required power, the thickness of the bottom of the puff support plate 13 may be made thin, and the transmission power of light may be increased to reach a side opposite to the UV lamps 20.

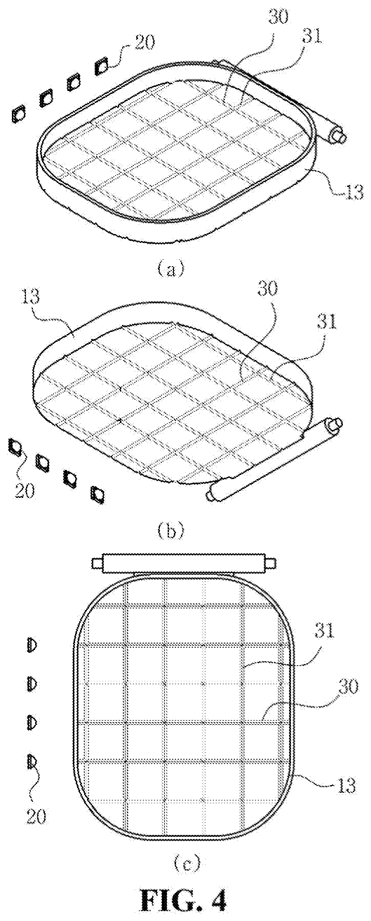

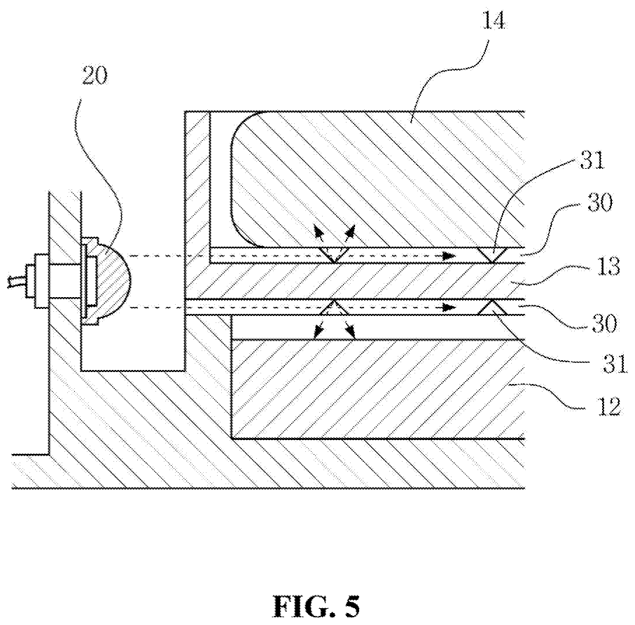

The present invention will be described with reference to FIGS. 4 and 5, as follows:

As shown in the drawings, in the configuration of the present invention, direct passage grooves 30 configured to pass the light of the UV lamps 20 therethrough along rectilinear lines may be formed in the bottom and top surfaces of the puff support plate 13.

Furthermore, there may further be formed orthogonal grooves 31 formed in a direction orthogonal to the direct passage grooves 30 and configured to transfer the light, transferred along the direct passage grooves 30, in the orthogonal direction.

The direct passage grooves 30 and the orthogonal grooves 31 may be formed as V-shaped grooves, as shown in the drawing. Although not shown in the drawing, the shape of the grooves may be formed as a rectangular, semi-circular, or polygonal shape, or the like.

In this manner, the light of the UV lamps 20 may be passed through the direct passage grooves 30 along rectilinear lines to a side opposite to the side on which the UV lamps 20 are mounted, and this light may be passed through the orthogonal grooves 31 to both side surfaces.

This case allows light to be almost completely transferred compared to a case using the light-guiding of the puff support plate 13. Accordingly, effects are achieved in that the overall contaminated surfaces are uniformly and strongly sterilized, and light transferred rectilinearly and orthogonally and light transferred using light-guiding are combined to maximize a sterilizing effect.

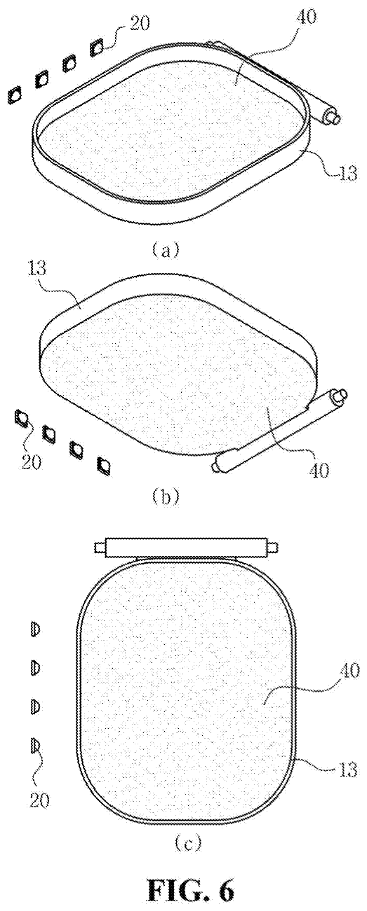

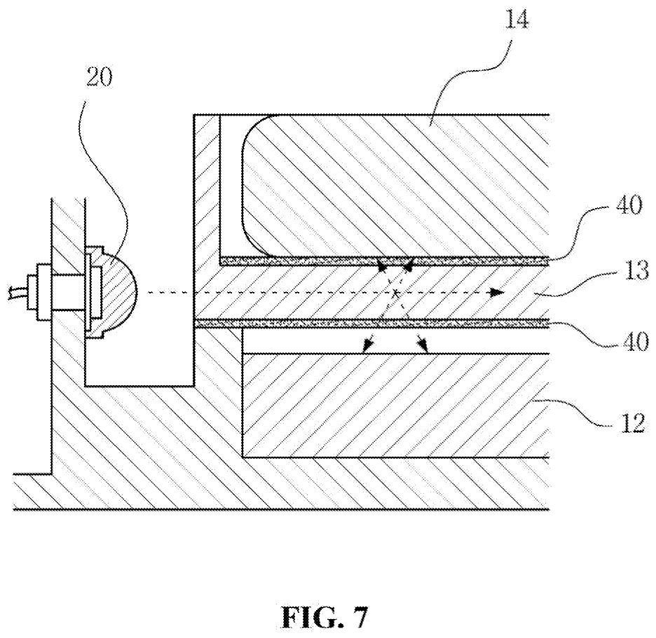

The present invention will be described with reference to FIGS. 6 and 7, as follows:

As shown in the drawings, in the configuration of the present invention., silver nano-pearls 40 configured such that sterilizing ions are emitted therefrom in response to the light of the UV lamps 20 may be formed on the bottom and top surfaces of the puff support plate 13.

The silver nano-pearls 40 may be formed by attaching silver nano-sheets, depositing silver nano-targets, applying a silver nano-paint, or the like.

It is preferable that the silver nano-pearls 40 be formed within a range that prevents the transparent or translucent puff support plate 13 from being made opaque.

As is well known, silver has an excellent antibacterial effect. When this silver is formed as a nano-type coating, it is not only inexpensive, but also provides an effect in that sterilizing ions are emitted in response to the light of the UV lamp 20 and thus improve sterilization and antibacterial ability along with ultraviolet rays.

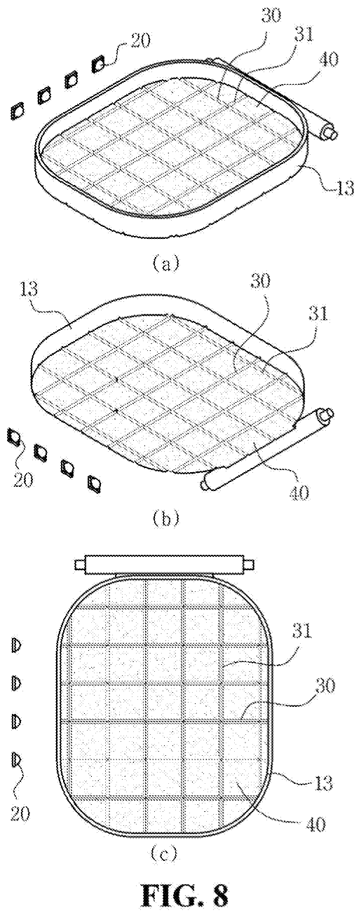

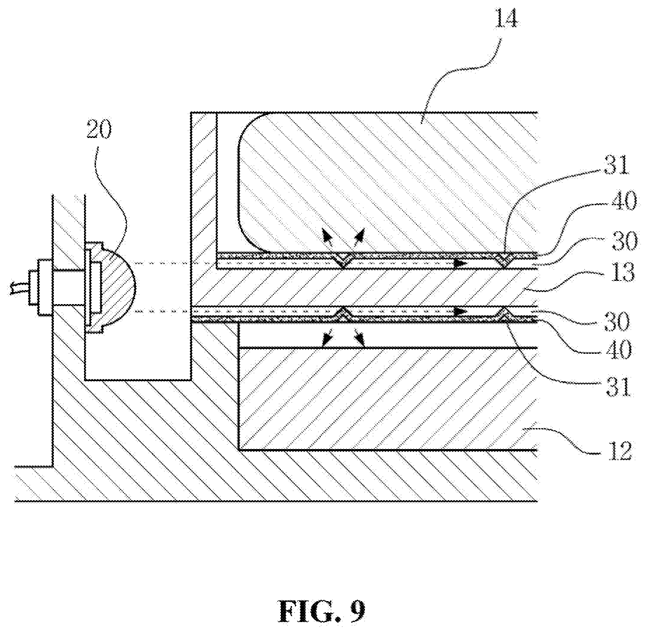

The present invention will be described with reference to FIGS. 8 and 9, as follows:

As shown in the drawings, in the configuration of the present invention, the direct passage grooves 30 configured to pass the light of the UV lamps 20 therethrough along rectilinear lines and the orthogonal grooves 31 formed in a direction orthogonal to the direct passage grooves 30 and configured to transfer the light, transferred through the direct passage grooves 30, in the orthogonal direction are formed on the bottom and top surfaces of the puff support plate 13, and the silver nano-pearls 40 configured such that sterilizing ions are emitted in response to the light of the UV lamps 20 therefrom are formed on the bottom and top surfaces of the puff support plate 13 on which the direct passage grooves 30 and the orthogonal grooves 31 are formed.

In this manner, the light of the UV lamps 20 is almost completely transferred via the direct passage grooves 30 and the orthogonal grooves 31, and also sterilizing ions are emitted. Accordingly, the sterilizing ions are emitted in response to the almost complete light rather than light through the light-guiding of the putt support plate 13, and thus the effect of emitting sterilizing ions relay be maximized. In addition, this is combined with the light through the light guiding of the puff support plate 13 to further maximize a sterilizing effect.

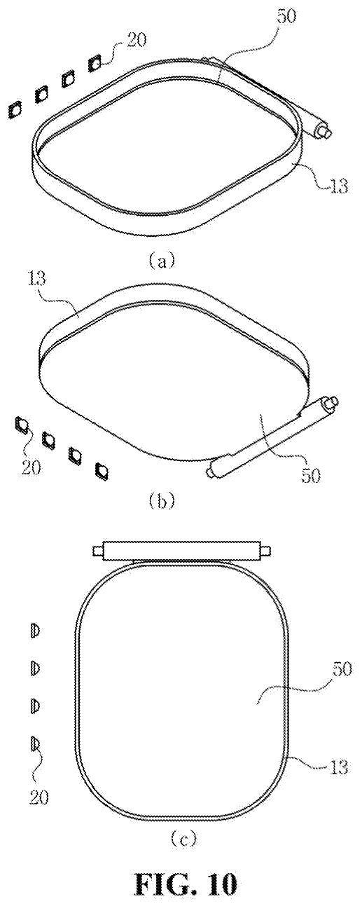

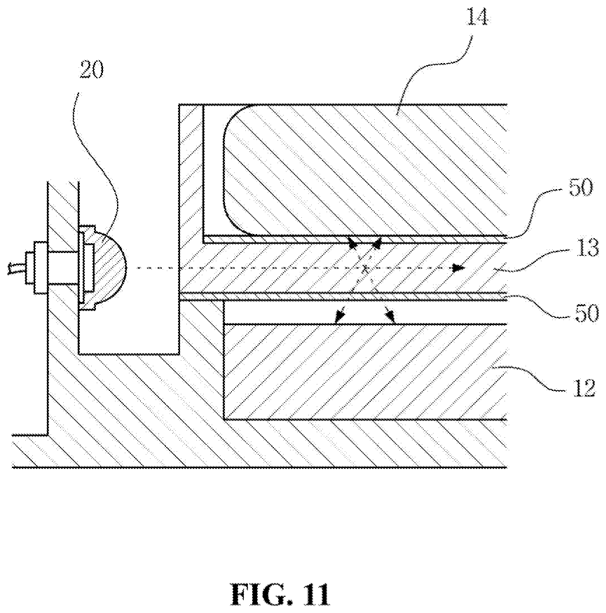

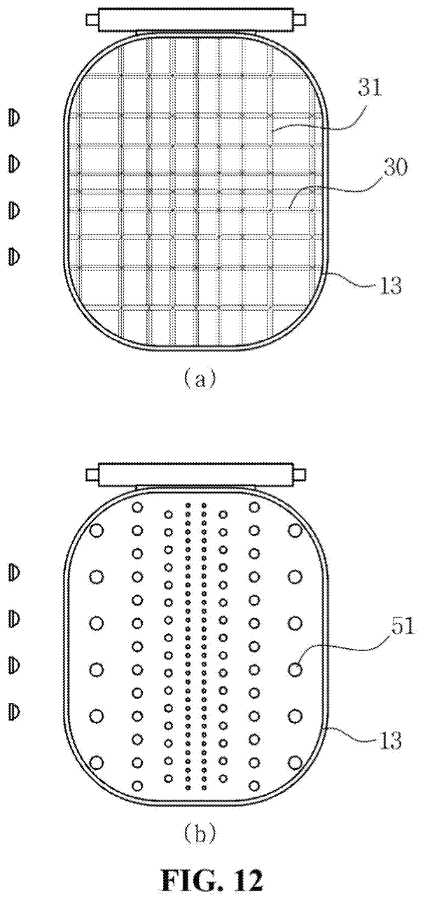

The present invention will be described with reference to FIGS. 10 to 12, as follows:

As shown in the drawings, in the configuration of the present invention, separate light guide plates 50 may be attached to the bottom and top, surfaces of the puff support plate 13.

When the light guide plates 50 are attached, the puff support plate 13 may be formed transparent, and the light guide plate 50 may be formed transparent or translucent.

In this manner, it is easy to apply the light guide plate 50, and also it is not necessary to manufacture the puff support plate 13 in the form of the light guide plate 50, thereby providing the effect of improving the convenience of manufacturing.

Furthermore, any one of the direct passage grooves 30, the orthogonal grooves 31, convex or concave circular or polygonal diffusion patterns 51, and the silver nano-pearls 40 may be formed on each of the light guide plates 50.

In this manner, it is not necessary to form the direct passage grooves 30, the orthogonal grooves 31, the diffusion patterns 51, or the silver nano-pearls 40 directly on the puff support plate 13. Accordingly, effects are achieved in that the convenience of the manufacturing of the puff support plate 13 is improved and it is easy to manufacture the light guide plate 50 having the above-described configuration.

Furthermore, the direct passage grooves 30 and the orthogonal grooves 31 formed in the light guide plates 50 may be formed to have narrower intervals toward the centers of the puff support plate 13.

In this manner, the light transferred via the direct passage grooves 30 and the orthogonal grooves 31 is concentrated on the centers of the puff support plate 13, i.e., contamination-concentrated surfaces, and thus an effect is achieved in that the contamination-concentrated surfaces may be intensively sterilized.

Furthermore, the diffusion patterns 51 formed on the light guide plate 50 may be formed to have narrower intervals and smaller sizes toward the centers of the puff support plate 13.

in this manner, the light transferred through the light guide plates 50 is diffused via the diffusion patterns 51, and the diffusion is concentrated in the centers of the puff support plate 13, i.e., contamination-concentrated surfaces, and thus an effect is achieved in that the contamination-concentrated surfaces may be intensively sterilized.

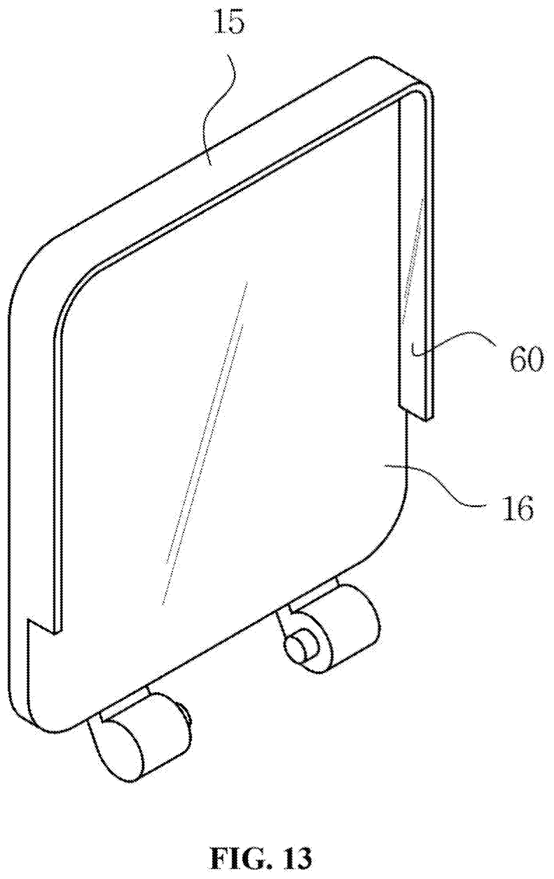

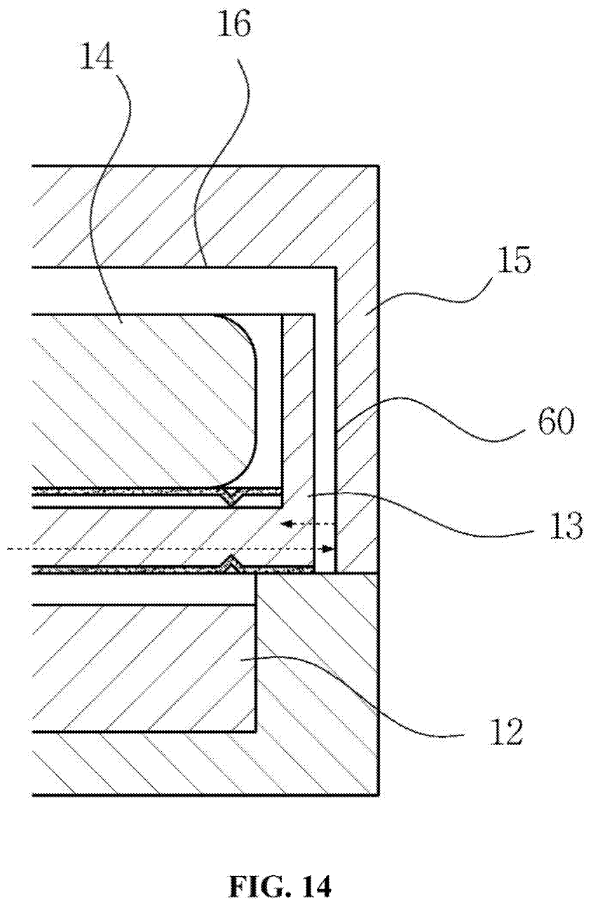

The present invention will be described with reference to FIGS. 13 and 14, as follows:

As shown in the drawings in the configuration of the present invention, the inner circumferential surface of the cover 15 may be formed as a reflector 60 configured to reflect the light transferred by the UV lamps 20.

In this manner, the light radiated by the UV lamps 20 an red to an opposite side or a side surface through the bottom of the puff support plate 13, the direst passage grooves 30, and the orthogonal grooves 31 may be reflected inward by the reflector 60, and thus an effect is achieved in that sterilizing ability may be further improved by maximizing the diffusion of light.

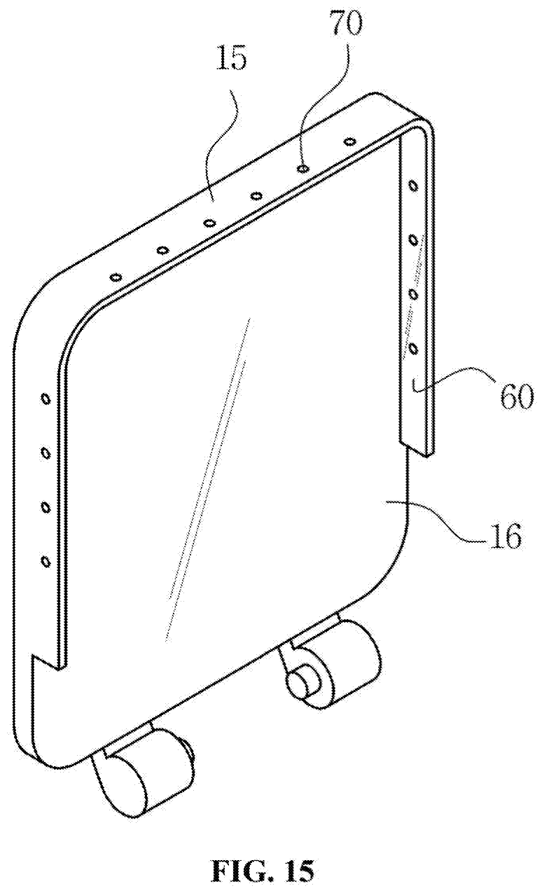

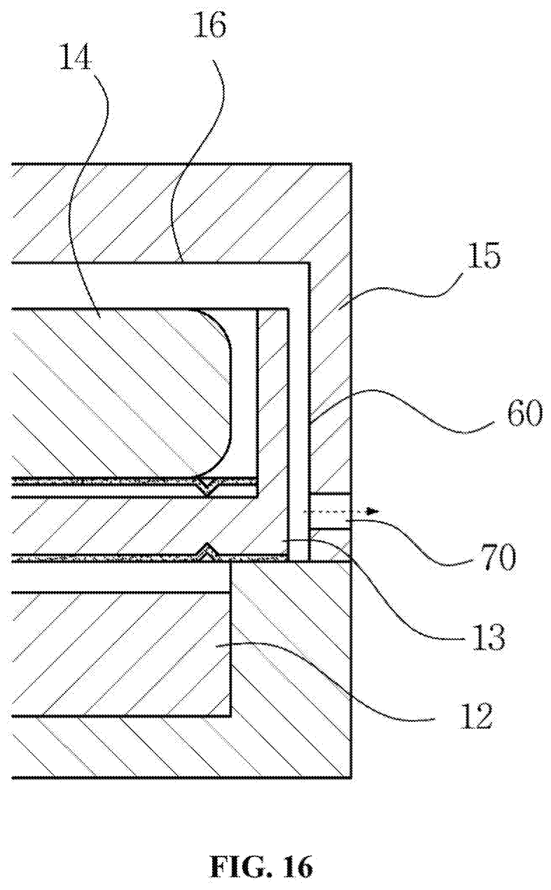

The present invention will be described with reference to FIGS. 15 and 16, as follows:

As shown in the drawings, in the configuration of the present invention, small holes 70 configured to the light transferred by the UV lamps 20 to be recognized from the outside and to allow air to enter or exit may be formed along the inner circumferential surface of the cover 15.

In this manner, effects are achieved in that a user may easily recognize the operating status of the UV lamps 20, the design may be made more aesthetic by the light emitted during the operation of the UV lamps 20, inside temperature may be prevented from rising by venting heat generated during the operation of the UV lamps 20 to the outside, and a contaminated surface may be dried by appropriate air communication.

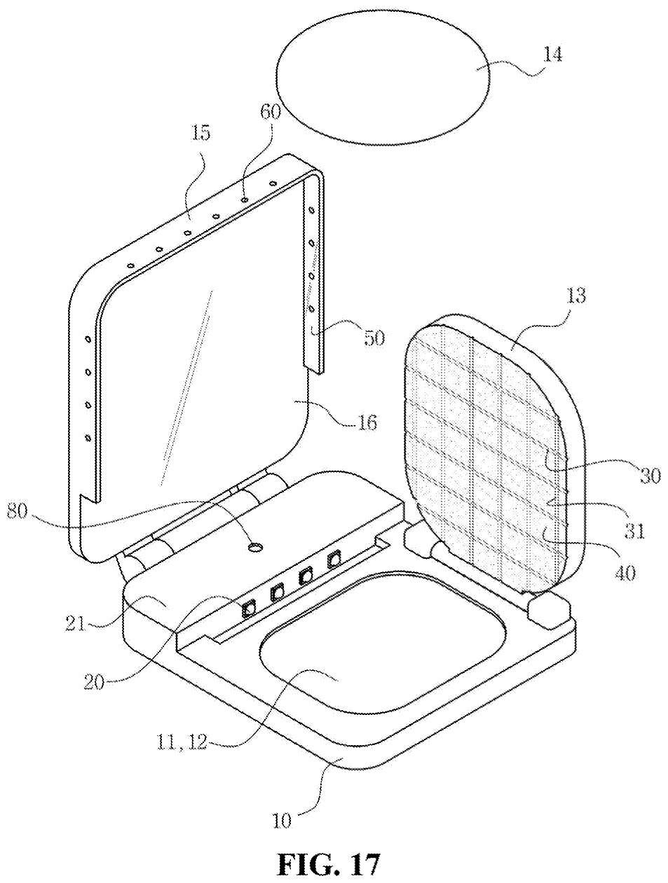

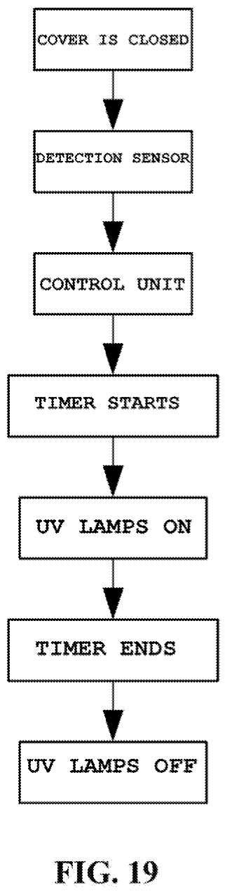

The present invention will be described with reference to FIGS. 17 to 19, as follows:

As shown its in the drawings, in the configuration of the present invention, there may be included a detection sensor 80 configured to detect the closing of the cover 15 on the top surface of the lamp device portion 21 from a substrate 22 configured to drive the UV lamps 20, a timer 81 configured to measure the time when the cover 15 is closed by the detection of the detection sensor 80, and a control unit 82 configured to automatically control the driving time of the UV lamps 20 by means of the timer 81.

In other words, the control unit 82 allows the timer 81 to be started through the detection of the detection sensor 80 when the cover 15 is closed, starts sterilization by turning on the UV lamps 20 for the time set by the timer 81, and automatically ends the sterilization by turning off the UV lamps (20) when the set timer (81) is terminated.

In this manner, effects are achieved in that there is provided the convenience in which a user does not need to manually drive the UV lamps 20, unnecessary power consumption is prevented by performing sterilization only for an appropriate period, and the cosmetic 12 and the puff 14 may be prevented from being subjected to excessive drying and deterioration attributable to the heat of light emitted from the UV lamps 20 by the driving of the excessive UV lamps 20.

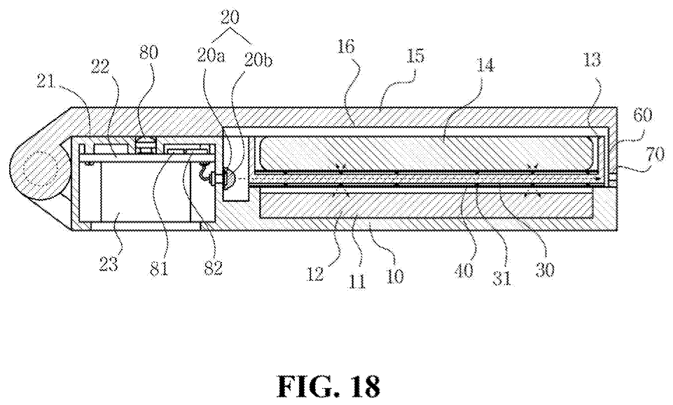

For reference, FIGS. 17 to 18 are perspective and sectional views in which all of the above-described configurations except for those of FIGS. 10 to 12 are merged together, and the configuration of the merged state and the effects thereof may be understood by referring to the drawings.

This completes the description of the present invention. In order to help understanding of the principle of the configurations to be pursued in the present invention, the configurations of the present invention and their specific components included in the configurations are illustrated, and the description of the present invention is made based on the drawings. The structures, shapes, patterns, arrangements, directions, and quantities of the compositions included in the present invention and their specific components are determined by considering the principle to be pursued, and various changes may be made as required. The configurations and their specific components suggested in the present invention are examples of the principles that are most desirably applied in order for those skilled in the art to obtain effects from the present invention and obtain better effects from the former effects. Accordingly, it is most preferable to complete the present invention by including all of the above-described configurations therein. However, due to cost reduction, the ease of manufacture, environmental conditions or needs, the present invention may be completed by selecting or excluding some of the configurations described above, or it may also be possible to separate one or some of the configurations separately and merge them with other configurations to complete the present invention. Furthermore, each configuration described above may be applied independently to other technical fields other than this technical field by considering a principle, a use, a function, a role, an action, an effect, or the like. Based on these, the scope of the present invention is specified as the scope that covers the claims of the present invention as follows, and the claims are determined and claims in order from claims having wider scopes. Through this, it seems that those having ordinary skill in the art may sufficiently understand the subject matter to be pursued in the present invention through the above-mentioned specific contents and the claims to be described below. The effects of the parts that are illustrated but not described will be sufficiently inferred through the drawings. Therefore, those skilled in the art may apply various modifications and changes in the technical field based on the contents mentioned in the present invention, and as a result, it may be possible to further increase the efficiency in use as well as the development of the technical field.

What is claimed is:

1. A UV sterilization device of a cosmetic case wherein:
in a cosmetic case including: a main body (10) including a cosmetic material accommodation space (11) filled with a cosmetic material (12), a puff support plate (13) disposed above the cosmetic material accommodation space (11) and mounted to be rotatable from one side of a top surface of the cosmetic material accommodation space (11), and a puff (14) configured to be seated on the puff support plate (13); a cover (15) mounted to be rotatable from one side of the main body (10) and to cover the maw body (10); and a mirror (16) disposed on n inner surface of the cover (15),
a lamp device portion (21) capable of driving UV lamps (20) is provided on one side of the main body (10), the cosmetic material accommodation space (11) is disposed in a lower portion of a remaining side of the main body (10), the puff support plate (13) is disposed above the cosmetic material accommodation space (11), the puff support plate (13) is formed transparent or translucent, the cosmetic material accommodation space (11) is formed opaque, and the UV lamps (20) are mounted to face from a side surface of the lamp device portion (21) to a bottom of the puff support plate (13),
the UV lamps (20) include UV-LEDs (20a), the UV-LEDs (20a) are covered with lenses (20b) capable of narrowing a radiation angle of the UV-LEDs (20a), direct passage grooves (30) configured to pass light of the UV lamps (20) therethrough along rectilinear lines are formed in bottom and top surfaces of the puff support plate (13), and orthogonal grooves (31) formed in a direction orthogonal to the direct passage grooves (30) and configured to transfer the light, transferred along the direct passage grooves (30), in the orthogonal direction are formed,
silver nano-pearls (40) configured such that sterilizing ions are emitted therefrom in response to the light of the lamps (20) are formed on the bottom and top surfaces of the puff support plate (13),
direct passage grooves (30) configured to pass light of the UV lamps (20) therethrough along rectilinear lines are formed in bottom and top surfaces of the puff support plate (13), orthogonal grooves (31) formed in a direction orthogonal to the direct passage grooves (30) and configured to transfer the light, transferred along the direct passage grooves (30), in the orthogonal direction are formed, and silver nano-pearls (40) configured such that sterilizing ions are emitted therefrom in response to the light of the UV lamps (20) are formed on the bottom and top surfaces of the puff support plate (13) which the direct passage grooves (30) and the orthogonal grooves (31) are formed,
separate light guide plates (50) are attached to the bottom and top surfaces of the puff support plate (13),
any one of the direct passage grooves (30), the orthogonal grooves (31), convex or concave circular or polygonal diffusion patterns (51), and the silver nano-pearls (40) are formed on each of the light guide plates (50),
the direct passage grooves (30) and the orthogonal grooves (31) formed in the light guide plates (50) are formed to have narrower intervals toward centers of the puff support plate (13),
the diffusion patterns (51) formed on the light guide plate (50) are formed to have narrower intervals and smaller sizes toward the centers of the puff support plate (13),
an inner circumferential surface of the cover (15) is formed as a reflector (60) configured to reflect the light transferred by the UV lamps (20),
small holes (70) configured to allow the light transferred by the lamps (20) to be recognized from an outside and to allow air to enter or exit are formed along an inner circumferential surface of the cover (15),
a detection sensor (80) configured to detect closing of the cover (15) on a top surface of the lamp device portion (21) from a substrate (22) configured to drive the UV lamps (20), a timer (81) configured to measure a time when the cover (15) is closed by detection of the detection sensor (80), and a control unit (82) configured to automatically control a driving time of the UV lamps (20) by means of the timer (81) are included, and the control unit (82) allows the timer (81) to be started through detection of the detection sensor (80) when the cover (15) is closed, starts sterilization by turning on the UV lamps (20) for a time set by the timer (81), and automatically ends the sterilization by turning off the UV lamps (20) when the set timer (81) is terminated, and
one or more of the above-described configurations are included.
